SEQUENCE LISTING (12 sequences)

SEQ ID NO: 1 - DNA, 19 bp, Artificial Sequence
  Description of Artificial Sequence Synthetic oligonucleotide
ctagcgttggcaccaccaa

SEQ ID NO: 2 - DNA, 20 bp, Artificial Sequence
  Description of Artificial Sequence Synthetic oligonucleotide
aatgttgggcagcgtccaca

SEQ ID NO: 3 - DNA, 14 bp, Artificial Sequence
  Description of Artificial Sequence Synthetic oligonucleotide
cgtccgctgatggg

SEQ ID NO: 4 - DNA, 30 bp, Artificial Sequence
  Description of Artificial Sequence Synthetic oligonucleotide
gtaagtgagatttagtctagggtaggtgac

SEQ ID NO: 5 - DNA, 24 bp, Artificial Sequence
  Description of Artificial Sequence Synthetic oligonucleotide
gggaccacctaccctacacctact

SEQ ID NO: 6 - DNA, 19 bp, Artificial Sequence
  Description of Artificial Sequence Synthetic oligonucleotide
tttggtctagggtaggccg

SEQ ID NO: 7 - DNA, 18 bp, Artificial Sequence
  Description of Artificial Sequence Synthetic oligonucleotide
atgaagaacgctgcgaac

SEQ ID NO: 8 - DNA, 22 bp, Artificial Sequence
  Description of Artificial Sequence Synthetic oligonucleotide
cagacatacttccaggcataac

SEQ ID NO: 9 - DNA, 20 bp, Artificial Sequence
  Description of Artificial Sequence Synthetic oligonucleotide
tcatcgaaattttgaacgca

SEQ ID NO: 10 - DNA, 16 bp, Artificial Sequence
  Description of Artificial Sequence Synthetic oligonucleotide
tcggcgaccggtttgt

SEQ ID NO: 11 - DNA, 20 bp, Artificial Sequence
  Description of Artificial Sequence Synthetic oligonucleotide
ccataccgcgaatcgaacac

SEQ ID NO: 12 - DNA, 15 bp, Artificial Sequence
  Description of Artificial Sequence Synthetic oligonucleotide
cggcgtttaatggag

(19) United States
(12) Patent Application Publication
(10) Pub. No.: US 2022/0162666 A1
(43) Pub. Date: May 26, 2022

(54) Soil Pathogen Testing

(71) Applicant: WinField Solutions, LLC, Arden Hills, MN (US)

(72) Inventors: Elizabeth Buescher, Saint Paul, MN (US); Clifford G. Watrin, Wyoming, MN (US); Marcus Jones, Ankeny, IA (US)

(21) Appl. No.: 17/534,890

(22) Filed: Nov. 24, 2021

Related U.S. Application Data
(60) Provisional application No. 63/117,885, filed on Nov. 24, 2020.

(51) Int. Cl. C12Q 1/06

(52) U.S. Cl. CPC C12Q 1/06; C12Q 2563/107; C12Q 2531/113

(57) ABSTRACT

A method of detecting pathogens within a soil sample involves extracting DNA from two or more pathogens within the soil sample. The pathogens include soybean cyst nematodes and one or more specimens of Phytophthora, Pythium, and/or Fusarium. The method further involves mixing the extracted DNA with a reagent mixture comprising a DNA polymerase, a mixture of deoxynucleotide triphosphates, two or more nucleic acid primer pairs each configured to bind with a target DNA sequence specific to one of the two or more pathogens, and two or more fluorophore-linked probes each configured to bind with a target DNA sequence specific to one of the two or more pathogens. The method subsequently involves amplifying each target DNA sequence via a quantitative polymerase chain reaction and quantifying each target DNA sequence by monitoring a fluorescence level of each of the two or more fluorophores.

### CROSS REFERENCE TO RELATED APPLICATIONS

This application claims priority to U.S. Provisional Application No. 63/117,885, filed Nov. 24, 2020, entitled “SOIL PATHOGEN TESTING” which is incorporated by reference herein, in its entirety and for all purposes.

### SEQUENCE LISTING

The instant application contains a Sequence Listing which has been submitted electronically in ASCII format and is hereby incorporated by reference in its entirety. Said ASCII copy, created on Nov. 23, 2021, is named P287700_US_02_SL.txt and is 3,041 bytes in size.

### TECHNICAL FIELD

Implementations relate to DNA-based soil pathogen detection and quantification for agricultural operations. Specific implementations involve the extraction of DNA from soil-borne pathogens present within a soil sample, and the subsequent amplification of two or more target DNA sequences via multiplex quantitative real-time polymerase chain reaction.

### BACKGROUND

Robust plant growth is critical to the success of commercial farming operations. To ensure robust growth, farming operations have been improved on several fronts. For example, many plant varieties have been genetically modified to enhance growth and yield; irrigation systems have been optimized; fertilizers have been formulated to compensate for particular nutrient deficiencies in specific climates; and, the assortment of pesticides, herbicides and other compositions typically applied to plants have been refined. Despite these improvements, the presence of soil-borne pathogens continues to damage nascent plants, thereby stunting growth and lowering overall yields. Preexisting techniques for detecting soil-borne pathogens may involve visual inspection of plants, which may be followed by the application of one or more pesticides formulated to eliminate the detected pathogens at the field locations from which the observations were made. While such techniques may be relatively easy and inexpensive to perform, new quantitative approaches are needed to increase the accuracy, efficiency, and throughput for pathogen detection that use soil samples collected for nutrient analysis.

### BRIEF DESCRIPTION OF THE DRAWINGS

FIG. 1 is a flow diagram of a method performed in accordance with principles of the present disclosure.

FIG. 2 is a qPCR standard curve usable for soybean cyst nematode (Heterodera glycines) egg quantification obtained by implementing embodiments of the present disclosure.

FIG. 3 is a qPCR standard curve usable for sudden death syndrome (Fusarium virguliforme) quantification obtained by implementing embodiments of the present disclosure.

FIG. 4 is a qPCR standard curve usable for Pythium quantification obtained by implementing embodiments of the present disclosure.

FIG. 5 is a qPCR standard curve usable for Phytophthora quantification obtained by implementing embodiments of the present disclosure.

### SUMMARY

In accordance with embodiments of the present disclosure, a method of simultaneously detecting pathogens within a soil sample can involve extracting DNA from two or more pathogens within the soil sample. The two or more pathogens can comprise or be selected from the group consisting of: soybean cyst nematode, a Phytophthora specimen, a Pythium specimen, and a Fusarium virguliforme specimen. The method may involve mixing the extracted DNA with a reagent mixture comprising a DNA polymerase, a mixture of deoxynucleotide triphosphates, two or more nucleic acid primer pairs each configured to bind with a target DNA sequence specific to one of the two or more pathogens, and two or more fluorophore-linked probes, each probe configured to bind with a target DNA sequence specific to one of the two or more pathogens. The method may further involve amplifying each target DNA sequence between each of the two or more nucleic acid primer pairs via a quantitative polymerase chain reaction. The method may further involve quantifying each target DNA sequence by monitoring a fluorescence level of each of the two or more fluorophores.

In some examples, one of the nucleic acid primer pairs can comprise, in a 5′-3′ direction: CTAGCGTTGGCACCACCAA and AATGTTGGGCAGCGTCCACA. In some embodiments, one of the nucleic acid primer pairs can comprise, in a 5′-3′ direction: GTAAGTGAGATTTAGTCTAGGGTAGGTGAC and GGGACCACCTACCCTACACCTACT. In some examples, one of the nucleic acid primer pairs can comprise, in a 5′-3′ direction: ATGAAGAACGCTGCGAAC and CAGACATACTTCCAGGCATAAC. In some examples, one of the nucleic acid primer pairs can comprise, in a 5′-3′ direction: TCGGCGACCGGTTTGT and CCATACCGCGAATCGAACAC. In some embodiments, the two or more nucleic acid primer pairs may further comprise at least one primer pair configured to bind to an internal control sequence. In some examples, at least one of the two or more fluorophore-linked probes can be configured to bind to the amplified DNA sequence via a probe sequence comprising, in a 5′-3′ direction: CGTCCGCTGATGGG, TTTGGTCTAGGGTAGGCCG, TCATCGAAATTTTGAACGCA, or CGGCGTTTAATGGAG. In some examples, the two or more nucleic acid primer pairs included in a given multiplex reaction can each be provided at a concentration of about 150 μM to about 250 μM, or any concentration therebetween, including for example 160 μM, 170 μM, 180 μM, 190 μM, 200 μM, 210 μM, 220 μM, 230 μM, or 240 μM. In some embodiments, concentrations lower than 150 μM may also be used, including for example 100 μM, 110 μM, 120 μM, 130 μM, or 140 μM.

In some embodiments, quantifying each target DNA sequence comprises determining an absolute quantity each target DNA sequence. In some examples, quantifying each target DNA sequence comprises determining a relative quantity of each target DNA sequence. In some embodiments, the quantitative polymerase chain reaction comprises an initial DNA denaturation step followed by 40 to 50 repeated cycles of DNA denaturation, DNA extension and DNA annealing. In some examples, each cycle of DNA denaturation can be performed at about 95° C. for about 15 seconds to about 60 seconds, each cycle of DNA annealing can be performed at about 58° C. to about 62° C. for about 15 seconds to about 60 seconds, and each cycle of DNA extension can be performed at about 72° C. for about 15 seconds to about 60 seconds. In some examples, the two or more nucleic acid primer pairs are each provided at a concentration of about 150 μM to about 250 μM.

In some examples, the method may also involve applying one or more pesticides to a field from which the soil sample was collected after quantifying each target DNA sequence within the soil sample. In some embodiments, the method also involves adjusting management practices such as a planting scheme in a field from which the soil sample was collected after quantifying each target DNA sequence within the soil sample. In some examples, the soil sample can be collected by a plant grower or soil-sampling service in a field. In some embodiments, the method further involves transmitting the soil sample to a remote laboratory before extracting DNA from two or more pathogens within the soil sample. In some examples, the two or more pathogens may comprise or consist of soybean cyst nematode and a Fusarium virguliforme specimen.

In accordance with embodiments of the present disclosure, a qPCR kit or assay for simultaneously detecting two or more soil-borne pathogens within a DNA sample may include: a DNA polymerase, a mixture of deoxynucleotide triphosphates, and two or more nucleic acid primer pairs each configured to bind with a target DNA sequence specific to one of the two or more soil-borne pathogens. The qPCR kit can also include two or more fluorophore-linked probes, each probe configured to bind with a target DNA sequence specific to one of the two or more pathogens and a volume of nuclease-free water. The two or more soil-borne pathogens can comprise or be selected from the group consisting of: soybean cyst nematode, a Phytophthora specimen, a Pythium specimen, and a Fusarium virguliforme specimen.

In some embodiments, the two or more soil-borne pathogens consist of soybean cyst nematode and a Fusarium virguliforme specimen. In some examples, the qPCR kit can also include at least one plasmid containing an internal control sequence. In some embodiments, one of the nucleic acid primer pairs included in the kit can comprise, in a 5′-3′ direction: CTAGCGTTGGCACCACCAA and AATGTTGGGCAGCGTCCACA. One of the nucleic acid primer pairs can comprise, in a 5′-3′ direction: GTAAGTGAGATTTAGTCTAGGGTAGGTGAC and GGGACCACCTACCCTACACCTACT. In some embodiments, the two or more soil-borne pathogens include one or more Pythium species and/or one or more Phytophthora species. In some examples, the qPCR kit can also include at least one plasmid containing an internal control sequence. In some embodiments, one of the nucleic acid primer pairs included in the kit can comprise, in a 5′-3′ direction: ATGAAGAACGCTGCGAAC and CAGACATACTTCCAGGCATAAC. One of the nucleic acid primer pairs can comprise, in a 5′-3′ direction: TCGGCGACCGGTTTGT and CCATACCGCGAATCGAACAC.

### DETAILED DESCRIPTION

Systems, methods, and reagents for simultaneously detecting the presence and quantity of multiple pathogens typically found in soil are disclosed herein. Implementations incorporate novel quantitative real-time polymerase chain reaction (“qPCR”) protocols and reagents configured for amplifying multiple DNA targets at the same time (“multiplex qPCR”). By simultaneously testing for multiple pathogens of varying size in a single qPCR reaction using DNA extracted from only one soil sample, the disclosed methods may increase testing efficiency, accuracy, and sensitivity relative to preexisting approaches implemented in agricultural settings. The soil sample used for pathogen detection may also be used for nutrient analysis, thereby further streamlining soil testing operations and improving downstream soil treatment approaches implemented by commercial growers. By surveying for soil-borne pathogens via DNA detection, pathogen assessments may also be performed throughout the year, not just when the pathogens are prevalent or dormant. Notably, the disclosed detection methods may be configured to simultaneously detect and quantify DNA collected from a wide range of genera and species, both macro and micro, marking a significant improvement over preexisting techniques. Despite their potential implementation across a greater number of cropping systems on a more frequent basis, the improved efficiency and accuracy of pathogen detection achieved via the disclosed techniques may also reduce the overall costs incurred by growers.

Preexisting approaches typically require separate sampling events for each species of pathogen due to the type of testing required for each pathogen. For example, soybean cyst nematode detection is often performed by counting nematode eggs under a microscope. Soil samples for nematode detection are usually collected during a collection window spanning from plant maturity up until plant harvest, during which the nematode count is frequently the highest and soil collection the easiest. Usually, only a single nematode sample is taken per several acres of farmland, which often misrepresents the true nematode prevalence at the site. Also, most soybean farmers only evaluate soybean cyst nematodes every three to five years, usually before a soybean crop rotation. The absence of commercial tests for any of the soil-borne pathogens disclosed herein, even for small-scale farming operations, further contributes to sparse testing practices, and because they are at least simple to implement and inexpensive on a per-test basis, growers have continued adhering to preexisting approaches, like manual counting. Plant growers unwilling or unable to implement a comprehensive pathogen detection plan have even chosen to bypass testing altogether in favor of precautionary pesticide application, which often leads to pesticide resistance.

Multiplex qPCR techniques have been used to simultaneously detect multiple DNA targets in other contexts. Such techniques are limited, however, to specific DNA sources, genetic targets, laboratory settings and qPCR platforms inapplicable to the systems and methods described herein, which can be implemented on a large, commercial scale. For example, preexisting methods are limited to specific pathogen types, e.g., infectious agents such as species of Giardia and Cryptosporidium, which have substantially similar physical properties, e.g., size, that make them more amenable to simultaneous extraction and amplification. In addition to the absence of flexible, non-target-specific protocols applicable to farm-based pathogen detection, the lack of soil-based qPCR systems may be attributed at least in part to the anticipated difficulty, expense, and specialized equipment necessary to extract and analyze multi-species DNA from soil samples collected at various timepoints in various geographic locations.

Development of the multiplex qPCR methods disclosed herein was hindered by the large disparities in pathogenic DNA concentrations collected within each soil sample. Deciphering which qPCR results reflected actual differences in DNA concentration and which qPCR results merely reflected preferential detection of one or more pathogens was difficult, especially given that soil sampling was not limited to a particular soil type or time of year. The simultaneous collection and detection of DNA from multiple distinct species was further complicated by differences in DNA organization specific to each species. Not all pathogens detected herein are equally amenable to DNA extraction. For example, it was not clear whether cell lysis techniques effective for one species would be equally effective for a different species, especially when the species are in different forms at different life stages at the point of collection. Non-specific binding of the qPCR primers also inhibited reliable quantification of the genetic targets discussed herein.

The disclosed methods, systems and reagents may be specific to soil-borne pathogens, i.e., non-human pathogens. As used herein, soil-borne pathogens include any bacteria, fungi, oomycete, algae, macro-pest and/or microorganism capable of causing a plant-based disease or otherwise harming plant roots, plant stems, plant leaves, plant flowers, and/or other plant parts, along with the soil in which plants are grown. Specific examples may include pathogenic species of Phytophthora spp. and/or Pythium spp. Pathogens responsible for causing various plant-based diseases, such as sudden death syndrome (primarily caused by Fusarium virguliforme), can also be detected according to embodiments described herein. Soil-borne pathogens may also include fully developed parasites, such as soybean cyst nematodes, capable of damaging plant tissue and/or stunting plant growth, which impacts yield. As used herein, the soil-borne pathogens may include specimens of one or more of the aforementioned pathogenic species. Each specimen may include individual microorganisms, collections of microorganisms, partially or fully developed parasitic and/or infectious organisms, pathogenic biomasses, and/or pathogenic bioproducts, such as eggs. By simultaneously analyzing pathogens regardless of identity, size and/or developmental stage, the disclosed methods achieve an enhanced level of comprehensiveness and flexibility relative to preexisting techniques, which typically rely on separate tests to detect DNA from species having such wide-ranging characteristics.

In specific embodiments, the two or more pathogens detected in a single test may include soybean cyst nematodes and specimens of Fusarium virguliforme. Some embodiments can detect one more additional pathogens and/or different combinations of pathogens.

As noted above, the pathogens targeted by the disclosed methods may include species of the Fusarium genus, e.g., Fusarium virguliforme, species of the Phytophthora genus, species of the Pythium genus, and/or soybean cyst nematodes (Heterodera glycines). Fusarium virguliforme is a soil-borne fungus that causes sudden death syndrome in a variety of crops, including soybeans, which leads to significant, widespread reductions in yield. Species of Phytophthora include oomycetes that cause crown and root rot diseases in a variety of plants, including many herbaceous and woody species, agricultural crops, fruit trees, nut trees and shrubs. In some advantageous examples, one species of Phytophthora, e.g., Phytophthora sojae, may be quantified and used as a proxy for all Phytophthora species present in a given soil sample. Pythium is a genus of parasitic oomycetes that causes multiple plant diseases, many of which lead to significant yield losses in an assortment of crops, especially herbaceous varieties. Similar to Phytophthora detection, in some examples, only one species of Pythium, e.g., Pythium ultimum, may be quantified and used as a proxy for all Pythium species present in a given soil sample. Soybean cyst nematodes are parasitic roundworms that also diminish yields by penetrating soybean roots and eventually accessing the vascular tissue and roots.

The disclosed methods may be implemented on a high-throughput basis in connection with various cropping systems. For example, the methods may be utilized to detect and reduce soil-borne pathogens present in the soil and/or on various plant types, including but not limited to: corn, soybeans, wheat, cotton, alfalfa, barley and potatoes. Soil samples may be collected from various geographical regions and from fields used for a variety of purposes. For example, soil may be collected from fields used for commercial farming operations. Soil may also be collected from locations not utilized for farming, such as plots subject to aesthetic planting and/or residential or commercial development. The commercially-scalable methods, kits and assays described herein may be configured specifically for small-, medium-, and/or large-scale farming operations, as opposed to epidemiological studies traditionally conducted in academic or private research settings. Embodiments may include collection devices and associated instructions necessary for farmers to sufficiently sample their field plots and ultimately determine whether certain pathogenic organisms pose a risk to their crops. Embodiments may also include mechanisms for reporting pathogenic results to farmers, such that the lab-based techniques are customized and integrated with farming operations. As such, the disclosed methods, kits, and assays may enable farmers to accurately assess the presence of pathogens within their fields in a manner not practical, affordable, or even attainable using preexisting approaches.

DNA detection and quantification achieved via the disclosed methods refers to the use of multiplex qPCR to amplify targeted DNA sequences within a mixture of genomic DNA extracted from two or more pathogenic species present within a soil sample, which may be derived from a commercial field plot. In general terms, the methods disclosed herein may involve obtaining at least one soil sample from at least one portion of a remote field location, extracting, and purifying the total genomic DNA from the soil sample, mixing the extracted DNA with a combination of qPCR reagents, and performing one or more qPCR reactions using a quantitative thermocycler machine with fluorimeter capabilities. The specific extraction and amplification parameters disclosed herein, along with the prophetic variations derived therefrom, were obtained and validated via extensive experimentation. The DNA amplification results may then be analyzed and delivered to a plant grower, who may subsequently implement one or more field-based measures in response to the presence, absence and/or amount of pathogens present within the soil sample. In some examples, the results may be reported to the plant grower in the form of a map tailored to the grower's field plot(s).

DNA Extraction

One or more soil samples can be collected from a location of interest, which as mentioned above, may include a field used for farming operations. In some examples, the soil samples may be collected from multiple fields and/or multiple locations within one field. Because of the advantageously small soil size required in some embodiments (e.g., less than 500 mg for fungal pathogens) and the high throughput of the methods disclosed herein, multiple soil samples can be collected and processed quickly, regardless of soil type and moisture level. Accordingly, soil samples having a range of sand, silt, clay, peat, and organic matter, among other substances, may be collected by plant growers or contractors tasked with collecting samples for lab analysis. In some embodiments, no specialized collection equipment is needed. Alternative embodiments may include collection devices, which may include one or more tools used to obtain the soil from varying depths, at least one container for depositing and transmitting the soil samples, and/or instructions for collecting the soil. Specialized tools may be provided to plant growers as part of a commercial kit. The depth at which a soil sample is collected may vary, ranging from about 1 inch or less from the surface, to about 2 inches, about 3 inches, about 4 inches, about 5 inches, about 6 inches, or more, or any depth therebetween. In some examples, a column of soil may be collected that spans from the soil surface to any of the aforementioned depths. The disclosed qPCR techniques can be advantageously employed to detect and quantify various soil-borne pathogens regardless of the soil sampling and/or DNA extraction techniques implemented.

In some embodiments, the soil collector(s) may select particular pathogens for testing. The pathogen(s) can be selected individually, as a subset, or as a full panel of pathogens. Pathogen selection can be made via paper, webpage, and/or using a cellular application customized for soil pathogen detection methods disclosed herein. In some examples, pathogen selection can be merged with one or more field maps, such that a plant grower can select certain pathogens for testing in certain field plots and/or locations within one or more field plots. Soil samples may be obtained and analyzed regardless of the presence or absence of foliar and/or root symptoms indicative of the presence of one or more pathogens. The methods disclosed herein may thus be implemented as a preventative measure in addition to, or instead of, a diagnostic measure implemented after planting and/or after suspected proliferation of one or more pathogens.

DNA extraction may be performed using a variety of techniques. Generally, genomic DNA can be extracted from soil-borne pathogens according to a multi-stage process that include: lysis, DNA precipitation, DNA binding, washing, elution and/or resuspension. In some embodiments, DNA extraction may be performed according to the methods described in U.S. application Ser. No. 16/855,589 and/or U.S. Patent Publication No. US2020/0123528A1, the entire contents of which are incorporated by reference herein. The disclosed extraction methods may have the versatility required to simultaneously extract DNA from a wide range of distinct genera and species without adjusting the extraction steps. DNA extraction may also be performed using a commercial kit, such as the DNeasy® PowerSoil® Kit sold by Qiagen and FastDNA™ Spin Kit sold by MP Biomedicals. In some embodiments, DNA extraction may not be implemented using a commercial kit. Such embodiments may involve extracting genomic DNA from pathogens having markedly different properties, such as size, which can necessitate customized extraction techniques that involve, for example, modified methods of cellular lysis.

In embodiments, lysis may involve mixing a soil sample with one or more enzymes, e.g., chitinase and/or cellulase, which may be utilized in addition to or in lieu of one or more mechanical lysis techniques, e.g., sonication, bead beating, freeze/thaw cycles, etc. To maximize the release of cellular contents, the soil sample may be processed prior to lysis by, for example, mixing the soil with water to form an aqueous slurry. Wet sieving may also be implemented for some soil samples, e.g., soil samples containing cysts. Dry soil samples ranging in mass from only about 250 mg to about 1 gram can be utilized in some embodiments, e.g., for fungal spore detection, although the methods described herein are not limited to a particular amount of soil. For soybean cyst nematode quantification, larger soil samples ranging from about 40 grams to about 200 grams, or more, may be required. At least one or more of the aforementioned techniques can be performed particularly when one or more amplification targets includes cysts, cyst eggs and/or fungal spores.

DNA precipitation may involve centrifuging the lysed cellular components and removing the supernatant for additional processing, which may involve mixing and incubating with isopropanol. An additional centrifugation step can be implemented to concentrate the DNA into a condensed pellet.

The precipitated DNA can then be isolated using one or more DNA binding steps, which may involve mixing the precipitated DNA with at least one buffer solution and one or more DNA-binding particles, such as silica or magnetic beads. The buffer solution can include guanidine thiocyanate (e.g., 6M) and water in some examples.

Lastly, one or more washing and/or elution steps may be implemented to remove non-DNA impurities, which can include soil debris, residual extraction reagents and cellular components. Various wash buffers can be utilized, which can include various amounts of sodium chloride, ethanol and/or water. The elution buffer can comprise a mixture of Tris-EDTA and water or just autoclaved water. Resuspension of the extracted DNA can be achieved by pipetting, shaking and/or vortexing immediately prior to DNA quantification and/or qPCR.

The extracted DNA may be quantified before qPCR. Various instruments may be utilized for quantification, including for example a DNA quantification plate reader, such as the SPECTROSTAR® Nano reader sold by BMG Labtech.

Multiplex qPCR

The multiplex qPCR assays and reagents described herein are designed to detect the presence and amount of at least two pathogens commonly present in soil. The disclosed methods may involve comparing the relative quantities of two or more pathogens determined via qPCR, which amplifies target DNA sequences through repeated cycles of DNA denaturation, annealing, and extension.

For qPCR, target-specific fluorescent reporters are used to monitor DNA amplification achieved after each amplification cycle, thereby revealing the absolute and/or relative amounts of target DNA present within a soil sample. In particular, the fluorescence tied to each target sequence may increase with each additional qPCR cycle until the fluorescence becomes measurable at a threshold cycle or crossing point (“Ct”). Lower Ct values for a target sequence mean that fewer amplification cycles were needed to produce measurable fluorescence, indicating a greater starting concentration of the targeted DNA within the original soil sample.

The specific qPCR parameters, e.g., denaturation temperature, annealing time, elongation time and/or reaction volume may vary depending on which qPCR platform is used. Parameters may also differ based on the number and/or type of pathogens targeted in a single assay. For example, greater reaction volumes may be utilized for qPCR protocols assaying four target genes versus qPCR protocols assaying only two target genes. Importantly, the qPCR parameters disclosed herein account for the preferential binding that may occur when the identity, size and/or starting concentrations of different DNA sequences are drastically different.

The disclosed multiplex qPCR assays were developed by designing and validating primers and fluorescent probes specific to each soil-borne pathogen, and implementing qPCR programs utilizing such reagents. The number of pathogens assayed in a reaction may vary. Embodiments may evaluate the presence and/or quantity of two, three, or four pathogens within a single test.

Generally, each qPCR reaction involves amplifying targeted DNA sequences (the “template DNA”) via a series of thermal cycling steps, each of which involves denaturing the template genomic DNA into two separate strands, hybridizing target-specific oligonucleotide primers thereto, and extending the hybridized primers using a thermostable DNA polymerase supplied with exogenous deoxynucleotide triphosphates (dNTPs). The dNTPs include equimolar amounts of guanosine deoxynucleotide triphosphate, cytosine deoxynucleotide triphosphate, adenosine deoxynucleotide triphosphate, thymidine deoxynucleotide triphosphate, and/or nucleotide analogs thereof. DNA polymerase (e.g., a Taq DNA Polymerase such as GoTaq®) is an enzyme configured to synthesize DNA strands from denatured template DNA using the supplied dNTPs. The synthesized primer sequences configured to flank each target sequence guide the DNA polymerase to each target site, thereby causing selective DNA amplification of each target sequence. Embodiments may thus involve initially mixing the extracted DNA with at least two primer sets, one set for each species-specific target sequence. In some examples, the dNTPs, DNA polymerase and one or more detection probes or dyes can be supplied together as a master-mix. In other examples, one or more of the aforementioned reagents may be added to a reaction mixture separately.

A qPCR procedure may be conducted immediately or substantially immediately after DNA extraction, or after a storage period implemented at about 4° C. (short-term storage) or about −20° C. (long-term storage). Accordingly, the extracted, purified DNA may require one or more initial preparation steps including but not limited to thawing, resuspending, diluting, and/or concentrating, for example.

The amount of template DNA utilized for a single qPCR reaction may vary and may depend on the amount or concentration of the DNA isolated via the extraction process. Based on the qPCR reactions performed and validated in accordance with this disclosure, the amount of DNA included in each reaction may range from about 0.1 to about 100 nanograms (ng), about 110 ng, about 120 ng, about 130 ng, about 140 ng, about 150 ng, or more than 150 ng. Examples can also utilize about 0.1 ng of DNA per reaction, or about 0.5 ng, about 1 ng, about 5 ng, about 10 ng, about 15 ng, about 20 ng, about 25 ng, about 30 ng, about 35 ng, about 40 ng, about 45 ng, about 50 ng, more than 50 ng, or any value therebetween. DNA samples having a concentration higher than a targeted range may be diluted with nuclease-free water. In solution, the volume of template DNA included in each reaction mixture may range from about 1 to about 20 μL, about 2 to about 16 μL, about 3 to about 12 μL, about 4 to about 10 μL, about 5 to about 8 μL, about 6 μL, about 8 μL, about 10 μL, about 12 μL, about 14 μL, about 16 μL, about 18 μL, about 20 μL, more than 20 μL, or any volume therebetween. In some embodiments, the volume of template DNA may be provided as a percentage of the total reaction volume, such as about 10%, about 15%, about 20%, about 25%, about 30%, about 35%, about 40%, about 50%, or any percentage therebetween. A greater number of amplification cycles may be necessary to amplify lower concentrations of template DNA. The length of qPCR may thus be tied at least in part to the effectiveness of DNA extraction and/or the quantity of pathogens present within a given soil sample.

The concentration and volume of each primer pair included in each reaction mixture was determined after extensive investigation into potential off-target and preferential primer binding. The primer pairs can include a first pair configured to hybridize to opposite strands of a targeted DNA sequence specific to a first pathogen, and a second pair configured to hybridize to opposite strands of a targeted DNA sequence specific to a separate pathogen. Each additional pathogen targeted by a single qPCR reaction will add another primer pair to the reaction mixture, each primer pair configured to flank a unique DNA sequence. The concentration of each primer pair may vary, ranging from about 100 nM to about 900 nM, or less than 100 nM, or about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, greater than 900 nM, or any concentration therebetween. The volume of each primer pair added to a given reaction mixture may range from less than 0.5 to about 0.5 μL, about 1.25 μL, about 1.5 μL, about 1.75 μL, about 2.0 μL, about 2.5 μL, about 3.0 μL, about 3.5 μL, about 4.0 μL, greater than 4.0 μL, or any volume therebetween.

Each primer pair includes a forward primer sequence configured to bind upstream of a target sequence and a reverse primer sequence configured to bind downstream of the same target sequence. In this manner, each primer pair flanks a target sequence specific to a species of interest, e.g., soybean cyst nematode. In some embodiments, the primer sequences used for soybean cyst nematode detection may be configured to hybridize within the organism's internal transcribed spacer sequence (“ITS”), which is a highly conserved region of genomic DNA unique to the species. Based on the qPCR reactions performed and validated in accordance with this disclosure, a forward primer sequence for soybean cyst nematode detection may have a sequence identical or substantially identical to SEQ ID NO: 1, and a corresponding reverse primer sequence may be identical or substantially identical to SEQ ID NO: 2. A fluorophore-linked, amplicon-specific probe sequence for soybean cyst nematode detection may be identical or substantially identical to SEQ ID NO: 3. As defined herein, a substantially identical sequence may comprise a nucleotide sequence having at least 55%, 60%, 70%, 75%, 80%, 85%, 90%, 95%, 98%, or 99% identity to any of SEQ ID NOS: 1-3.

In some embodiments derived from the qPCR reactions performed and validated in accordance with this disclosure, the primer sequences used for Fusarium virguliforme detection (sudden death syndrome) may be configured to hybridize within the organism's multi-copy number intergenic spacer (“IGS”) region, which is a ribosomal DNA region specific to Fusarium species. A forward primer sequence for Fusarium virguliforme detection may have a sequence identical or substantially identical to SEQ ID NO: 4, and a corresponding reverse primer sequence may be identical or substantially identical to SEQ ID NO: 5. A fluorophore-linked, amplicon-specific probe sequence for Fusarium virguliforme may be identical or substantially identical to SEQ ID NO: 6. As defined herein, a substantially identical sequence may comprise a nucleotide sequence having at least 55%, 60%, 70%, 75%, 80%, 85%, 90%, 95%, 98%, or 99% identity to any of SEQ ID NOS: 4-6.

In some embodiments derived from the qPCR reactions performed and validated in accordance with this disclosure, the primer sequences used for Pythium detection may be configured to hybridize within a Pythium organism's (e.g., Pythium ultimum) multi-copy number internal transcribed spacer (“ITS”) region, which is a ribosomal DNA region specific to Pythium species. A forward primer sequence for Pythium detection may have a sequence identical or substantially identical to SEQ ID NO: 7, and a corresponding reverse primer sequence may be identical or substantially identical to SEQ ID NO: 8. A fluorophore-linked, amplicon-specific probe sequence for Pythium may be identical or substantially identical to SEQ ID NO: 9. As defined herein, a substantially identical sequence may comprise a nucleotide sequence having at least 55%, 60%, 70%, 75%, 80%, 85%, 90%, 95%, 98%, or 99% identity to any of SEQ ID NOS: 7-9.

In some embodiments derived from the qPCR reactions performed and validated in accordance with this disclosure, the primer sequences used for Phytophthora detection may be configured to hybridize within a Phytophthora organism's (e.g., Phytophthora sojae) multi-copy number internal transcribed spacer (“ITS”) region, which is a ribosomal DNA region specific to Phytophthora species. A forward primer sequence for Phytophthora detection may have a sequence identical or substantially identical to SEQ ID NO: 10, and a corresponding reverse primer sequence may be identical or substantially identical to SEQ ID NO: 11. A fluorophore-linked, amplicon-specific probe sequence for Phytophthora may be identical or substantially identical to SEQ ID NO: 12. As defined herein, a substantially identical sequence may comprise a nucleotide sequence having at least 55%, 60%, 70%, 75%, 80%, 85%, 90%, 95%, 98%, or 99% identity to any of SEQ ID NOS: 10-12.

The length of each target DNA sequence flanked by a primer pair was selected in part to minimize preferential amplification of one target sequence over another. The target sequence lengths (or amplicon lengths) were also selected to maximize the efficacy of qPCR amplification. For example, longer target sequences may be amplified less efficiently than shorter target sequences. The amplicon lengths targeted herein may range from less than about 60 base pairs (bp) to about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, about 190 bp, about 200 bp, greater than about 200 bp, or any length therebetween. The location of each target sequence within a genome may vary, and in some cases may lie within a conserved domain specific to a given pathogenic species.

At least one internal control construct, which can be a double-stranded DNA structure, can be included in each reaction to confirm the absence of qPCR inhibition. In some embodiments, the internal control construct may be included within an exogenous plasmid added to each qPCR mixture. The same control plasmid may be used for a variety of distinct multiplex reactions, in some implementations, thereby enabling the same “universal” control to be used for a variety of different reactions. Each internal control will be targeted by a unique pair of primers in the same manner as the species-specific DNA targets discussed above. A control construct can include a region configured to bind with an upstream primer of a target sequence and a region configured to bind with a downstream primer of a target sequence. The intervening body sequence may be substantially random but will be configured to bind with a fluorescent probe for detection. The number of unique internal control constructs may be the same as the number of DNA sequences targeted in a particular multiplex reaction. Detection of the internal control signifies that a given qPCR reaction is successfully amplifying target DNA using the primer pairs included in the reaction. The length of the non-primer-binding portion of the control construct may vary and may be similar to or substantially the same as the length of one or more target sequences.

A fluorescent probe or fluorophore may be used to enable real-time PCR amplification of a target sequence (“amplicon”) via detection of an increase in fluorescence signal. Some embodiments may utilize a fluorophore covalently attached to a target-specific probe, e.g., TaqMan probe, as mentioned above. According to such embodiments, as DNA replication occurs and dNTPs are added to the synthesized DNA strand, the reporter fluorescence is cleaved by the Taq polymerase, separating the reporter from the quencher and the reporter dye allowed to fluoresce. In some embodiments, the fluorophore can be a double-stranded DNA binding dye. According to such embodiments, the fluorescence signal increases as amplification proceeds and more double-stranded DNA is produced. Each primer pair may be tagged with a fluorescent probe or fluorophore having a unique emission spectrum. The fluorescent probe may be configured to hybridize with a region of the qPCR amplicon flanked by the primers. The final concentration of each probe may also vary, ranging from less than about 100 μM to about 100 about 150 about 200 about 250 about 300 about 350 about 400 about 450 about 500 μM, greater than 500 μM, or any concentration therebetween. The particular fluorophores used in a given reaction may be selected from an assortment of commercially available fluorophores. Example fluorophores (and their excitation maximum and emission maximum (in nanometers)) compatible with the qPCR programs implemented and validated according to the present disclosure include but are not limited to: fluorescein (490/513), Oregon Green (492/517), FAM (494/518), SYBR® Green (494/521), EvaGreen (500/530), TET (521/538), JOE (520/548), VIC (538/552), Yakima Yellow (526/552), HEX (535/553), Cy®3 (552/570), Bodipy® TMR (544/574), NED (546/575), TAMRA (560/582), ABY (568/583), Cy3.5 (588/604), ROX (587/607), Texas Red (596/615), JUN (606/618), LightCycler Red 640 (625/640), Bodipy 630/650 (625/640), Alexa Fluor 647 (650/666), Cy5 (643/667), Mustang Purple (647/654), Alexa Fluor 660 (663/690), and Cy5.5 (683/707).

After the necessary pre-processing steps have been performed to prepare the template DNA, the DNA may be admixed with one or more of the qPCR reagents noted above, which may be provided separately or together as a complete kit. The resulting reaction mixture may be aliquoted into separate wells of a multi-well plate, e.g., a 96- or 384-well plate. For some reactions utilizing a master-mix of qPCR reagents, only the template DNA may need to be added separately to each reaction well after the master-mix has been added, depending on the reagents included in the master-mix. Negative control wells may include master-mix and water but no DNA. Each unique reaction mixture may be included in duplicate or triplicate in the same multi-well plate to minimize the likelihood of obtaining statistically insignificant results. In some examples, a qPCR kit utilized to detect and quantify one or more of the pathogenic species disclosed herein, e.g., soybean cyst nematode, a Phytophthora specimen, a Pythium specimen, and/or Fusarium virguliforme, may include a DNA polymerase, a mixture of deoxynucleotide triphosphates (which may be equimolar), and two or more nucleic acid primer pairs each configured to bind with a target DNA sequence specific to one of the two or more soil-borne pathogens. The kit may also include two or more fluorophore-linked probes, each probe configured to bind with a target DNA sequence specific to one of the two or more pathogens. Nuclease-free water may also be included or added separately. In some particular embodiments, the two or more soil-borne pathogens detected using a prepackaged kit may include or consist of soybean cyst nematode and a Fusarium virguliforme specimen. Embodiments of the kit may include at least one plasmid containing an internal control sequence. In some particular embodiments of a kit, one of the nucleic acid primer pairs may include SEQ ID NOS: 1 and 2. In addition or alternatively, one of the nucleic acid primer pairs may include SEQ ID NOS: 4 and 5. In addition or alternatively, one of the nucleic acid primer pairs may include SEQ ID NOS: 7 and 8. In addition or alternatively, one of the nucleic acid primer pairs may include SEQ ID NOS: 10 and 11. Each of the aforementioned sequences, along with the corresponding probe sequences noted above, were validated by implementing embodiments of the extraction and multiplex amplification reactions disclosed herein.

Nuclease-free water may be added to reach a target volume, which may vary for different reactions utilizing different primer pairs or DNA templates. The total reaction volume for each qPCR assay may vary. In some examples, the total reaction volume may be about 10 μL, 12 μL, 15 μL, 18 μL, 20 μL, 25 μL, 30 μL, 35 μL, 40 μL, 45 μL, 50 μL or more, depending on the number of pathogens targeted in a single reaction. The reagent volume for each soil-borne pathogen, which includes one set of target-specific primers, may be about 0.5 μL.

After an initial DNA denaturation step, amplifying the targeted DNA sequences and internal controls is achieved via numerous repeated thermal cycling steps, each of which includes DNA denaturation, extension, and annealing. Based on the reactions performed in accordance with the present disclosure, the number of cycles implemented for a single qPCR assay may range from about 40 to about 50 cycles, including for example 40 cycles, 41 cycles, 42 cycles, 43 cycles, 44 cycles, 45 cycles, 46 cycles, 47 cycles, 48 cycles, 49 cycles, or 50 cycles. The particular qPCR parameters implemented in each cycle may depend on the qPCR platform utilized for the reaction. The methods and reagents disclosed herein may be compatible with an assortment of thermocycler machines. Example machine platforms that may be used include but are not limited to one or more systems sold by: Applied Biosystems®, Bio-Rad, Roche, Quantabio, Integrated DNA Technologies (IDT), Stratagene®, and/or Qiagen. In some examples, which may depend on the particular qPCR platform utilized, a uracil N-glycosylase (UNG) incubation step may be implemented prior to the initial denaturation. Specific, non-limiting examples may include a UNG incubation implemented at about 50° C. for about 2 minutes. Embodiments may include incubation temperatures ranging from about 40° C. to about 60° C., for example 42° C., 44° C., 46° C., 48° C., 52° C., 54° C., 56° C., 58° C., or any temperature therebetween. Incubation times may range from about 1 minute to about 3 minutes, for example about 1.5 minutes, 2 minutes, 2.5 minutes, or any length therebetween. Additionally, embodiments may include a polymerase activation step implemented between the UNG incubation and the initial denaturation. Specific, non-limiting examples may include a 2-minute polymerase activation step implemented at 95° C. Embodiments may include activation temperatures ranging from about 91° C. to about 99° C., or any value therebetween, including for example 92° C., 93° C., 94° C., 95° C., 96° C., 97° C., and 98° C. Activation times may range from about 1 minute to about 3 minutes, for example 1.5 minutes, 2 minutes, 2.5 minutes, or any length therebetween.

The initial denaturation step may be implemented at a range of temperatures for various lengths of time. In examples, the initial denaturation step may be performed at about 95° C. for about 1 minute, about 2 minutes, about 3 minutes, about 4 minutes, about 5 minutes, about 6 minutes, about 7 minutes, about 8 minutes, about 9 minutes, about 10 minutes, or longer. The initial denaturation step may be implemented only once.

The denaturation step implemented pursuant to each repeated thermal cycle may also be performed at a range of temperatures for various lengths of time. For example, repeated denaturation cycles may be performed at about 95° C. for about 10 seconds, about 15 seconds, about 20 seconds, about 25 seconds, about 30 seconds, about 35 seconds, about 40 seconds, about 45 seconds, about 50 seconds, about 55 seconds, about 60 seconds, or longer. Denaturation temperatures may also vary in some embodiments, ranging from about 91° C. to about 99° C., or any value therebetween, including for example 92° C., 93° C., 94° C., 95° C., 96° C., 97° C., and 98° C. In some particular examples, one or more denaturation steps may be performed at about 95° C. for about 15 seconds to about 60 seconds.

The annealing step included in each repeated thermal cycle may also be performed at a range of temperatures and periods of time. In various examples, the annealing step may be performed at about 56° C., 58° C., 59° C., 60° C., 61° C., 62° C., 63° C., 64° C., any value therebetween, or higher. The annealing step may last about 10 seconds, about 15 seconds, about 20 seconds, about 25 seconds, about 30 seconds, about 35 seconds, about 40 seconds, about 45 seconds, about 50 seconds, about 55 seconds, about 60 seconds, or longer. In some particular examples, one or more annealing steps may be performed at about 58° C. to about 62° C. for about 15 seconds to about 60 seconds.

The extension step included in each thermal cycle may be performed at about 68° C., 69° C., 70° C., 71° C., 72° C., 73° C., 74° C., 75° C., 76° C., any value therebetween, or higher. The extension step may last about 10 seconds, about 15 seconds, about 20 seconds, about 25 seconds, about 30 seconds, about 35 seconds, about 40 seconds, about 45 seconds, about 50 seconds, about 55 seconds, about 60 seconds, or longer. In some particular examples, one or more extension steps may be performed at about 72° C. for about 15 seconds to about 60 seconds. In some examples, one or more discrete extension steps may be excluded, such that only successive denaturation and annealing steps are performed.

The number of thermal cycling steps may depend on a variety of factors. For example, longer DNA target sequences, e.g., between about 120 and 200 bp, may require a greater number of amplification cycles. Specific embodiments may include a single initial denaturation step followed by 40-45 cycles of denaturation, annealing and extension. Specific embodiments may include 5 μL of master mix, 0.5 μL of each primer, up to 4 μL of DNA template, and a volume of nuclease-free water necessary to reach a total reaction volume of 10 μL. Another specific embodiment may include 10 μL of master mix, 1 μL of each primer, up to 8 μL of DNA template, and the volume of nuclease-free water necessary to reach a total reaction volume of 20 μL. Depending on its concentration, the volume of DNA template may vary, ranging from about 1 μL to about 2 μL, about 3 μL, about 4 μL, about 5 μL, about 6 μL, about 7 μL, more than 7 μL, or any volume therebetween. Highly concentrated DNA samples may be diluted to avoid amplification inhibition.

As noted above, the pathogens targeted by the qPCR assays disclosed herein may be selected by a user, e.g., plant grower. As such, customized qPCR reactions can be ordered to identify the presence and/or quantity of a specified set of pathogens. Embodiments may involve assaying a full panel of pathogens, or only a subset of interest.

The disclosed methods may be performed one or more times per year. For example, two or more soil-borne pathogens may be detected immediately before planting, one or more times after planting but before harvesting, immediately after harvesting, and/or one or more times between harvesting and planting. The soil may also be tested at one or more locations within the same field at one or more of the aforementioned times. According to such examples, soil samples may be collected from two more locations within a virtual grid overlaid on the field, for instance a 2.5-acre grid overlaid on a 20-acre sampling zone. Embodiments disclosed herein may be integrated into grid sampling practices, e.g., about 1- to about 20-acre grid sampling operations. Embodiments may additionally or alternatively be integrated into zone sampling practices, which involve sampling one or more unique zones within a field. Combining soil samples from similar soil textures may be critical in some embodiments. Testing at multiple locations may enable growers to optimize fertilizer and herbicide application within a single field plot to minimize pathogen proliferation without wasting fertilizers or herbicides and without increasing the likelihood of pesticide resistance.

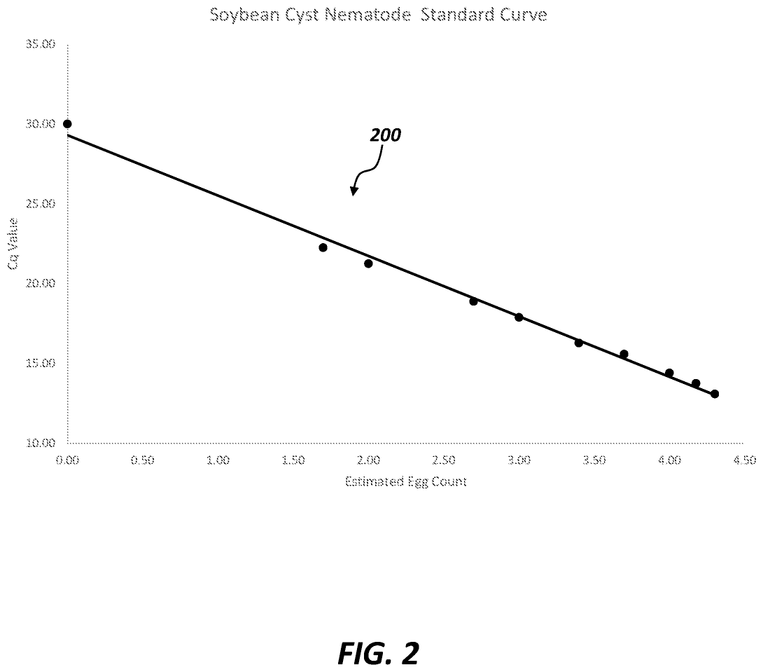

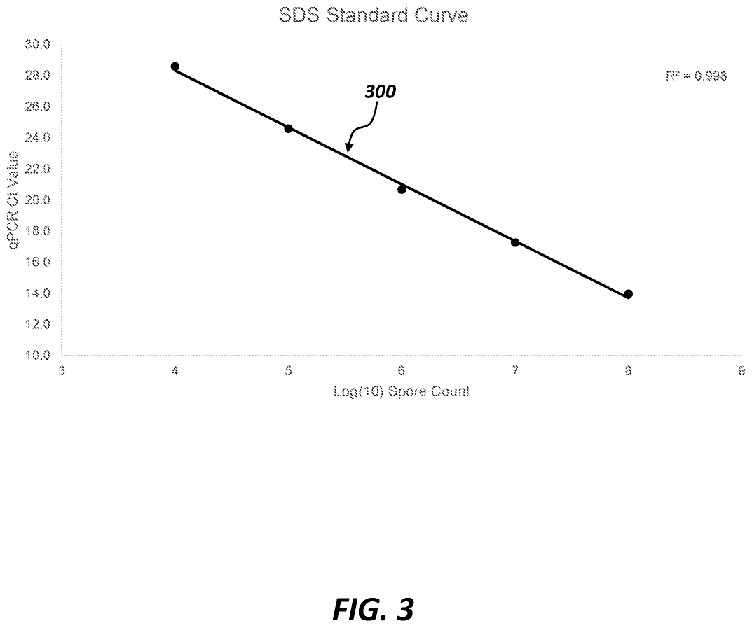

The DNA extraction and/or qPCR assays may be implemented at a lab facility remote from the soil collection site, and the results transmitted back to the growers in the form of a hard-copy report and/or a digital report viewable on a webpage or user interface, which can be displayed on a mobile device such as a phone, tablet, or laptop. Downstream analyses performed by a lab operator and/or plant grower may involve determining the absolute quantity or quantities of various pathogens and/or comparing the relative quantity or quantities of various pathogens. Absolute quantities of a given pathogen, which may be estimated using a standard curve generated for that pathogen (see FIGS. 2 and 3) may comprise the number of spores, eggs, DNA copies, etc. Using this information, a plant grower can apply one or more seed treatments, e.g., insecticides, fungicides and/or pesticides, and/or can plant seeds having one or more natural or genetically-engineered traits most conducive to plant growth in a particular soil-pathogen profile. Methods may also involve adjusting the pesticide compositions applied to the soil at the collection site. Planting schemes may also be adjusted at the collection site, for example such that non-host plants naturally resilient to a detected pathogen are planted at the site, while plants particularly sensitive to a detected pathogen are planted elsewhere. In this manner, plant waste and/or pesticide use is minimized. Repeated soil testing can give plant growers a detailed look at which pathogens are most prevalent at certain times of year and/or in certain locations throughout a given field, information that was not practically obtained using preexisting methods of visual soil observation. A plant grower may also modify the irrigation and/or drainage systems in response to the qPCR data. For example, many Phytophthora species thrive in warm, moist soils. Data showing moderate to high levels of Phytophthora in a soil sample may thus be used to decrease moisture levels at the collection site(s).

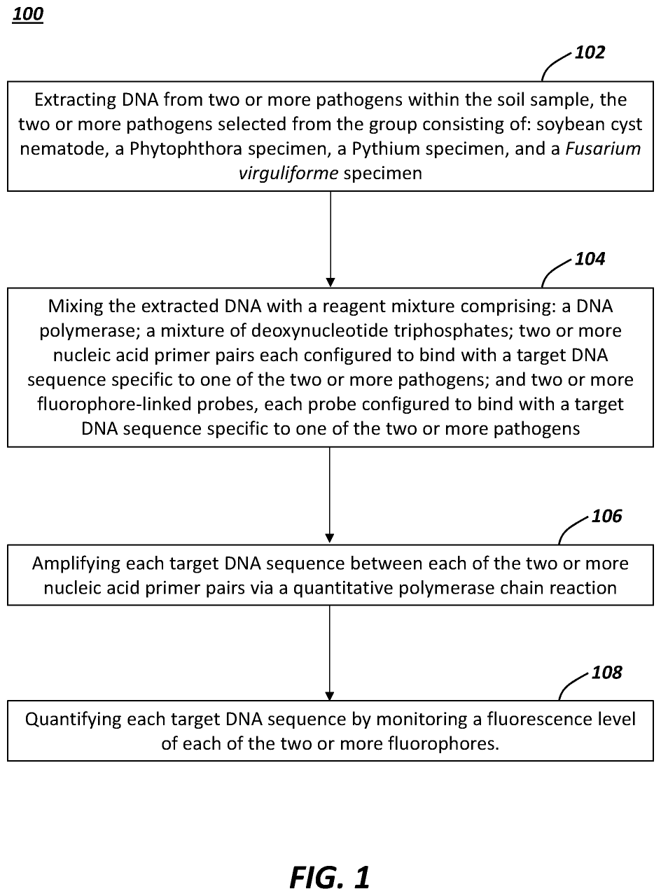

FIG. 1 shows an example method 100 of simultaneously detecting pathogens within a soil sample according to one or more embodiments described herein. The steps shown in FIG. 1 may be implemented in the order shown, or in a different order. For example, the steps may be performed sequentially in any order, contemporaneously, separately, together, or any combination thereof. In some examples, the steps shown in FIG. 1 must be performed in the order shown. Additional steps may be added in alternative embodiments.

The illustrated method 100 involves, at step 102, “extracting DNA from two or more pathogens within the soil sample, the two or more pathogens selected from the group consisting of: soybean cyst nematode, a Phytophthora specimen, a Pythium specimen, and a Fusarium virguliforme specimen.” Step 104 involves “mixing the extracted DNA with a reagent mixture comprising: a DNA polymerase; a mixture of deoxynucleotide triphosphates; two or more nucleic acid primer pairs each configured to bind with a target DNA sequence specific to one of the two or more pathogens; and two or more fluorophore-linked probes, each probe configured to bind with a target DNA sequence specific to one of the two or more pathogens.” At step 106, the method further involves “amplifying each target DNA sequence between each of the two or more nucleic acid primer pairs via a quantitative polymerase chain reaction,” and at step 108 the method involves “quantifying each target DNA sequence by monitoring a fluorescence level of each of the two or more fluorophores.”

To confirm the methods disclosed herein and create prophetic reference charts for quickly estimating the quantities of multiple pathogens present in a given soil sample, DNA extraction and multiplex qPCR was performed in accordance with method 100 and cycle threshold (“Ct”) values obtained for each pathogen. The generated reference charts, shown in FIGS. 2-5, can be utilized to accurately estimate pathogen quantities by simply implementing one or more methods disclosed herein. In particular, by extracting DNA and performing multiplex qPCR in accordance with the present disclosure, Ct values for one or more pathogens can be obtained and compared. Aligning the obtained Ct values with the estimated pathogen quantities on the Ct reference charts enables users to obtain reliable estimations of one or more pathogens within a given soil sample without the need for manual counting or observation.

The Ct values shown in the figures were obtained by extracting DNA from soil samples in the manner described herein and performing qPCR on the extracted DNA. The specific qPCR program implemented in this example included an initial 2-minute uracil-N-glycosylase (UNG) incubation step implemented at 50° C. and a 2-minute polymerase activation step implemented at 95° C., followed by 40 cycles of denaturation (each 1 second at 95° C.) and annealing/extension (each 20 seconds at 60° C.). A melt curve analysis was implemented by heating the reaction to 95° C. for 15 seconds, 60° C. for 1 minute, and 95° C. for 15 seconds. The primer and probe sequences consisted of SEQ ID NOS: 1-12. The total volume of the qPCR reaction volume was 10 which included 2 μL of DNA (˜40 ng), 5 μL of a (2×) master mix (containing dNTPs, DNA polymerase, buffer, reference dye), 0.5 μL of (20×) Taqman® assay, and 2.5 μL of nuclease-free water. Manual spore and egg count estimations were used to build the standard curve for Fusarium virguliforme and soybean cyst nematode, respectively. For each of Pythium and Phytophthora, genome copy number was estimated using the amount of DNA in each sample (in ng) and the length of the targeted DNA template (in base pairs) according to Equation 1.1:

$$\frac{Amount of DNA ( ng ) \times ( 6.022 \times {10}^{23} )}{Amplicon length ( bp ) \times ( 1 \times {10}^{9} ) \times 660} \qquad Equation 1.1$$

FIG. 2 shows the qPCR standard curve 200 developed by performing qPCR on defined samples of soil containing soybean cyst nematode (“SCN”) eggs. The standard curve 200 can be used as a reference to determine the quantity of SCN eggs within a soil sample after performing qPCR on the DNA extracted from the sample. In particular, by referencing the standard curve 200, a Ct value obtained via qPCR can be converted to an estimated quantity of SCN eggs present within a given soil sample. The standard curve 200 shows that a qPCR Ct value of about 29.5 indicates an egg count of 0 or below detection limits; a Ct value of about 25 indicates an egg count of about 16 (Calculation: 10^{1.2}); a Ct value of about 20 indicates an egg count of about 251 (Calculation: 10^{2.4}); and a Ct value of about 15 indicates an egg count of about 5623. Additional SCN egg counts can be estimated by aligning a newly obtained Ct value with a point along the curve 200.

FIG. 3 shows the standard curve 300 developed by performing qPCR on defined samples of soil containing Fusarium virguliforme spores (primary causal pathogen of sudden death syndrome or “SDS”) within a soil sample. By referencing the standard curve 300, a Ct value obtained via qPCR can be converted to an estimated quantity of SDS spores present within a given soil sample. The standard curve 300 shows that if a qPCR Ct value of about 14 is obtained, the SDS spore count is about 100,000,000. Additionally, a Ct value of about 17.5 indicates a spore count of about 10,000,000; a Ct value of about 20.5 indicates a spore count of about 1,000,000; a Ct value of about 24.5 indicates a spore count of about 100,000; and a Ct value of about 28.5 indicates a spore count of about 10,000. Additional SDS spore counts can be estimated by aligning a newly obtained Ct value with a point along the curve 300.

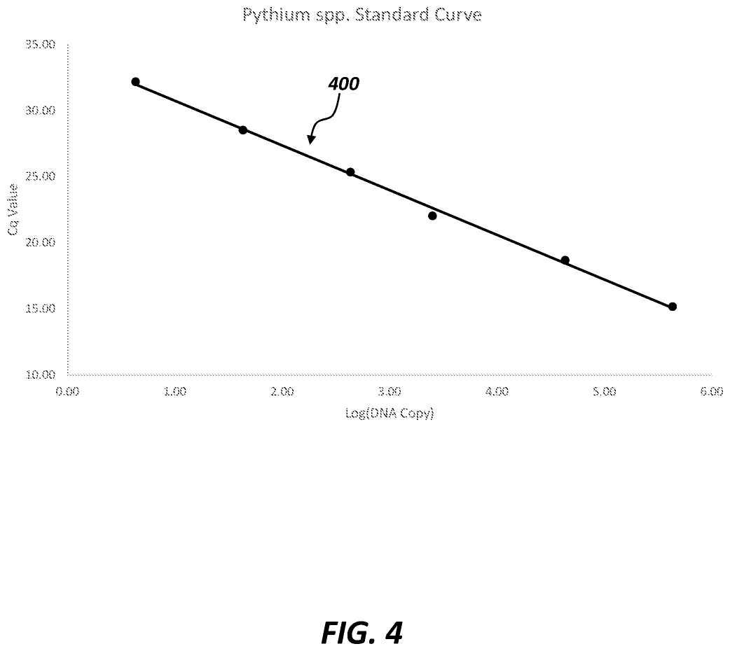

FIG. 4 shows the qPCR standard curve 400 developed by performing qPCR on defined samples of soil containing Pythium ultimum. The standard curve 400 can be used as a reference to determine the amount of all Pythium species within a soil sample after performing qPCR on the DNA extracted from the sample (Pythium ultimum can be used as a proxy to represent all genome sizes for Pythium species). In particular, by referencing the standard curve 400, a Ct value obtained via qPCR can be converted to an estimated copy number of Pythium genomes present within a given soil sample. The standard curve 400 shows that a qPCR Ct value of about 30 indicates a genome copy number of about 13 (Calculation: 10^{1.1}); a Ct value of about 25 indicates a genome copy number of about 501 (Calculation: 10^{2.7}); a Ct value of about 20 indicates a genome copy number of about 10000 (Calculation: 10⁴); and a Ct value of about 15 indicates a genome copy number of about 501187 (Calculation: 10^{5.7}). Additional Pythium genome copy numbers can be estimated by aligning a newly obtained Ct value with a point along the curve 400.

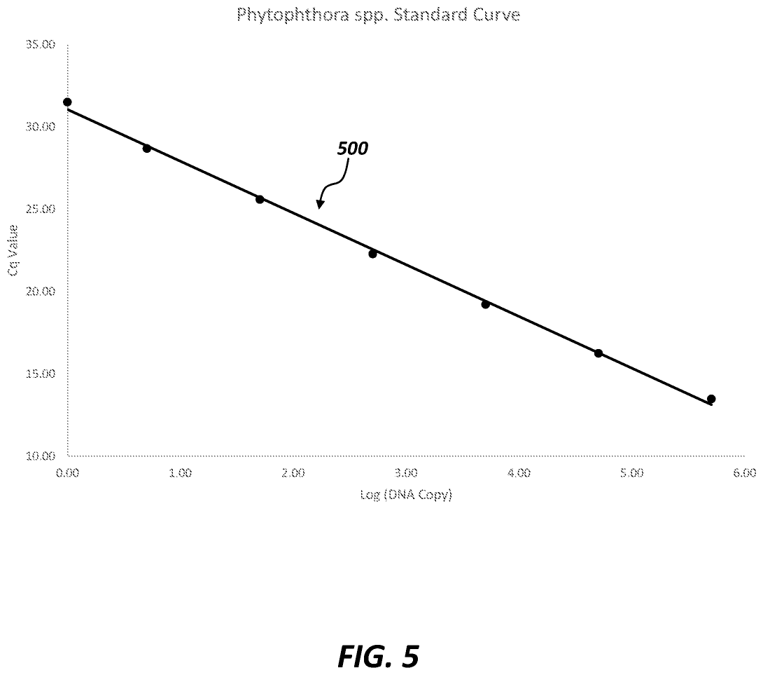

FIG. 5 shows the qPCR standard curve 500 developed by performing qPCR on defined samples of soil containing Phytophthora sojae. The standard curve 500 can be used as a reference to determine the amount of Phytophthora within a soil sample after performing qPCR on the DNA extracted from the sample (Phytophthora sojae can be used as a proxy to represent all genome sizes for Phytophthora species). In particular, by referencing the standard curve 500, a Ct value obtained via qPCR can be converted to an estimated copy number of Phytophthora genomes present within a given soil sample. The standard curve 500 shows that a qPCR Ct value of about 32 indicates a genome copy number of 0 or below detection limits; a Ct value of about 25 indicates a genome copy number of about 79 (Calculation: 10¹⁹); a Ct value of about 20 indicates a genome copy number of about 3162 (Calculation: 10¹⁵); and a Ct value of about 15 indicates a genome copy number of about 25893 (Calculation: 10^{5.1}). Additional Phytophthora genome copy numbers can be estimated by aligning a newly obtained Ct value with a point along the curve 500.

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or components used to carry out the methods, and like proximate considerations. The term “about” also encompasses amounts that differ due to aging of a formulation with a particular initial concentration or mixture and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

Although the present disclosure provides references to preferred embodiments, persons skilled in the art will recognize that changes may be made in form and detail without departing from the spirit and scope of the invention.

What is claimed is:

1. A method of simultaneously detecting pathogens within a soil sample, the method comprising:
extracting DNA from two or more pathogens within the soil sample, the two or more pathogens selected from the group consisting of: soybean cyst nematode, a Phytophthora specimen, a Pythium specimen, and a Fusarium virguliforme specimen;
mixing the extracted DNA with a reagent mixture comprising:
a DNA polymerase;
a mixture of deoxynucleotide triphosphates;
two or more nucleic acid primer pairs each configured to bind with a target DNA sequence specific to one of the two or more pathogens; and
two or more fluorophore-linked probes, each probe configured to bind with a target DNA sequence specific to one of the two or more pathogens;
amplifying each target DNA sequence between each of the two or more nucleic acid primer pairs via a quantitative polymerase chain reaction; and
quantifying each target DNA sequence by monitoring a fluorescence level of each of the two or more fluorophores.

2. The method of claim 1, wherein one of the nucleic acid primer pairs comprises:
5′-CTAGCGTTGGCACCACCAA-3′
5′-AATGTTGGGCAGCGTCCACA-3′

3. The method of claim 1, wherein one of the nucleic acid primer pairs comprises:
5′-GTAAGTGAGATTTAGTCTAGGGTAGGTGAC-3′
5′-GGGACCACCTACCCTACACCTACT-3′

4. The method of claim 1, wherein the two or more nucleic acid primer pairs further comprise at least one primer pair configured to bind to an internal control sequence.

5. The method of claim 1, wherein at least one of the two or more fluorophore-linked probes is configured to bind to the amplified DNA sequence via a probe sequence comprising:

| 5′-CGTCCGCTGATGGG-3′ | |
|---|---|
| or | |
| 5′-TTTGGTCTAGGGTAGGCCG | -3′. |

6. The method of claim 1, wherein quantifying each target DNA sequence comprises determining an absolute quantity each target DNA sequence.

7. The method of claim 1, wherein quantifying each target DNA sequence comprises determining a relative quantity of each target DNA sequence.

8. The method of claim 1, wherein the quantitative polymerase chain reaction comprises an initial DNA denaturation step followed by 45 to 50 repeated cycles of DNA denaturation, DNA extension and DNA annealing.

9. The method of claim 8, wherein each cycle of DNA denaturation is performed at about 95° C. for about 15 seconds to about 60 seconds, each cycle of DNA annealing is performed at about 58° C. to about 62° C. for about 15 seconds to about 60 seconds, and each cycle of DNA extension is performed at about 72° C. for about 15 seconds to about 60 seconds.

10. The method of claim 1, wherein the two or more nucleic acid primer pairs are each provided at a concentration of about 150 μM to about 250 μM.

11. The method of claim 1, further comprising applying one or more pesticides to a field from which the soil sample was collected after quantifying each target DNA sequence within the soil sample.

12. The method of claim 1, further comprising adjusting a planting scheme in a field from which the soil sample was collected after quantifying each target DNA sequence within the soil sample.

13. The method of claim 1, wherein the soil sample is collected by a plant grower in a field.

14. The method of claim 13, further comprising transmitting the soil sample to a remote laboratory before extracting DNA from two or more pathogens within the soil sample.

15. The method of claim 1, wherein the two or more pathogens consist of soybean cyst nematode and a Fusarium virguliforme specimen.

16. A qPCR kit for simultaneously detecting two or more soil-borne pathogens within a DNA sample, the qPCR kit comprising:
a DNA polymerase;
a mixture of deoxynucleotide triphosphates;
two or more nucleic acid primer pairs each configured to bind with a target DNA sequence specific to one of the two or more soil-borne pathogens;
two or more fluorophore-linked probes, each probe configured to bind with a target DNA sequence specific to one of the two or more pathogens; and
a volume of nuclease-free water,
wherein the two or more soil-borne pathogens are selected from the group consisting of: soybean cyst nematode, a Phytophthora specimen, a Pythium specimen, and a Fusarium virguliforme specimen.

17. The qPCR kit of claim 16, wherein the two or more soil-borne pathogens consist of soybean cyst nematode and a Fusarium virguliforme specimen.

18. The qPCR kit of claim 16, further comprising at least one plasmid containing an internal control sequence.

19. The qPCR kit of claim 16, wherein one of the nucleic acid primer pairs comprises:
5′-CTAGCGTTGGCACCACCAA-3′
5′-AATGTTGGGCAGCGTCCACA-3′

20. The qPCR kit of claim 19, wherein one of the nucleic acid primer pairs comprises:
5′-GTAAGTGAGATTTAGTCTAGGGTAGGTGAC-3′
5′-GGGACCACCTACCCTACACCTACT-3′
